(19) United States
(12) Patent Application Publication
(10) Pub. No.: US 2003/0191436 A1
(43) Pub. Date: Oct. 9, 2003

(54) Balloon catheter

(76) Inventor: Ronald Adrianus Maria Horvers, Geldrop (NL)

Correspondence Address: BARNARD & PAULY, P.S., P.O. Box 58888, Seattle, WA, 98138-1888, US

(21) Appl. No.: 10/117,922

(22) Filed: Apr. 5, 2002

(51) Int. Cl. A61M 29/00

(52) U.S. Cl. 604/103.04; 606/192

(57) ABSTRACT

The invention relates to a balloon catheter comprising a catheter tube having a first lumen and a second lumen, each lumen having a proximal end and a distal end, an inflatable balloon having a proximal end and a distal end, and a guide wire receivable in the second lumen. The distal end of the first lumen opens into the balloon. The distal end of the second lumen is open and positioned proximally of the proximal end of the balloon.

### TECHNICAL FIELD

The present invention relates to a balloon catheter comprising a catheter tube having a first lumen and a second lumen, each lumen having a proximal end and a distal end, an inflatable balloon having a proximal end and a distal end, and a guide wire receivable in the second lumen, with the distal end of the first lumen opening into the balloon.

### BACKGROUND INFORMATION

Such a balloon catheter is known, for example from U.S. Pat. Nos. 5,531,690; 5,496,346 and 4,762,129. With these known balloon catheters the guide wire extends through the second lumen, which extends alongside or through the balloon. The drawback of these known balloon catheters is the fact it is not easy to form a second lumen that extends through the balloon or abuts against the balloon. This may lead to problems, in particular in connection with the seal that is desired with a view to inflating the balloon. In addition to that, such a production method is relatively costly and the profile of the balloon is relatively high.

### SUMMARY OF THE INVENTION

It is an object of the present invention to provide a balloon catheter in which the above-mentioned drawbacks have been overcome. Another object of the present invention is to provide a balloon catheter in which the balloon catheter can be provided in a relatively inexpensive manner with a guide wire by means of which the balloon catheter can be guided through vessels and lumina of the human body.

In accordance with the present invention, the balloon catheter is characterized in that the distal end of the second lumen is open and positioned proximally of the proximal end of the balloon. As a result, the guide wire is led through the open distal end of the second lumen outside the balloon.

Preferably, the distal end of the balloon is connected with a tip for the purpose of strengthening the distal end of the balloon catheter.

Furthermore, the tip preferably comprises a lumen for receiving the distal end of the guide wire, by which a connection between the guide wire and the balloon catheter is obtained in a relatively simple manner.

In order to obtain a so-called Rapid Exchange type balloon catheter, the catheter is provided with an opening which is in communication with the second lumen, which opening is positioned proximally of the distal end of the second lumen.

In a preferred embodiment, the balloon catheter furthermore comprises a stiffening wire, which is received within the first lumen. Said stiffening wire provides the distal end of the balloon catheter with the required stiffness for being passed through vessels and lumina of the human body. Thereby, relatively more force can be applied to the tip as the stiffening wire extends into the distal end of the balloon catheter. This increases pushability of the balloon catheter.

In addition to that, the invention relates to a catheter having a proximal end and a distal end, comprising an inflatable balloon at the distal end of the catheter and a guide wire at least at the distal end of the catheter, wherein according to the invention the guide wire is positioned free of and outside of the balloon at the distal end of the catheter in the inflated condition of the balloon.

### BRIEF DESCRIPTION OF THE DRAWINGS

In the drawings, like element designations refer to like parts throughout, and:

FIG. 1 is a perspective view of a preferred embodiment of a balloon catheter according to the invention in inflated condition;

FIG. 2 shows the balloon catheter of FIG. 1 in non-inflated condition;

FIG. 3 is a sectional view of a balloon comprising a stiffening wire and a guide wire, which illustrates the manner in which the balloon catheter of FIG. 1 is obtained by means of a first folding method; and

FIG. 4 is a sectional view like FIG. 3, but showing a second folding method.

### BEST MODE FOR CARRYING OUT THE INVENTION

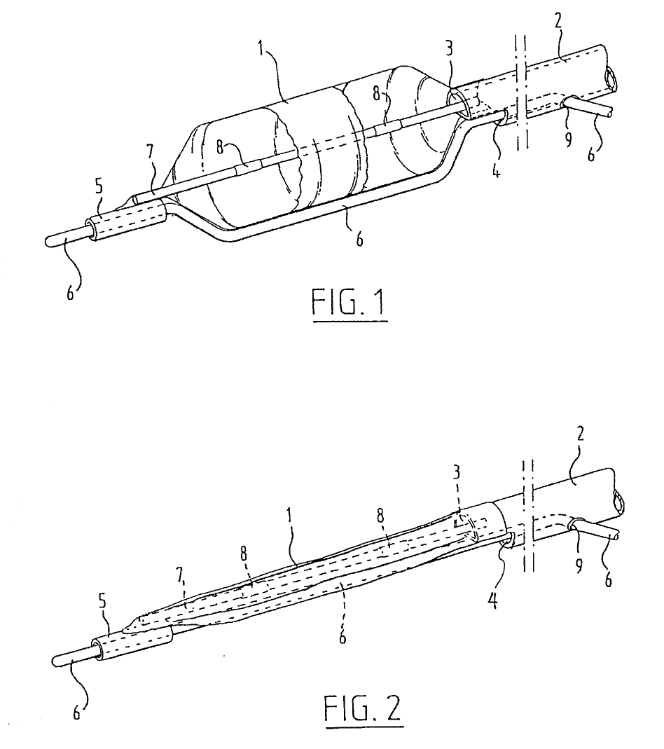

The perspective view of FIG. 1 shows a balloon catheter according to the invention with the balloon 1 in inflated condition. The balloon catheter comprises a catheter tube 2 having a first lumen 3 and a second lumen 4, each lumen 3, 4 having a proximal end and a distal end. The distal end of the first lumen 3 opens into the balloon 1. The inflatable balloon 1 likewise has a proximal end and a distal end, the proximal end being fixed to the catheter tube 2 and the distal end being fixed to a tip 5. The balloon catheter furthermore comprises a guide wire 6 to be received in the second lumen 4 and a stiffening wire 7 received within the first lumen 3. The stiffening wire 7 is provided with marking bands 8, which are discernible on x-ray equipment. The stiffening wire 7 extends from the first lumen 3, through the open distal end thereof and through the balloon 1, into the distal end of said balloon. This stiffening wire 7 is fixed to the tip 5 together with the distal end of the balloon 1.

The distal end of the second lumen 4 is open, and it is positioned proximally of the proximal end of the balloon 1. An opening 9, which is in communication with the second lumen 4, is furthermore provided in the catheter tube 2. Said opening 9 is positioned proximally of the distal end of the second lumen 4. The guide wire 6 can be passed through the opening 9 in the catheter tube 2 into the second lumen 4, and exit the second lumen 4 through the open distal end thereof. The guide wire 6 extends outside of the balloon 1 and is passed through the lumen of the tip 5.

In an inflated condition of the balloon 1 (FIG. 1), the guide wire 6 is positioned free of and outside of the balloon 1 between the open distal end of the second lumen 4 and the tip 5. In a non-inflated condition of the balloon 1 (FIG. 2), the guide wire 6 is still positioned outside of the balloon, but it is received within a cavity that has been obtained by folding the balloon according to a special folding method.

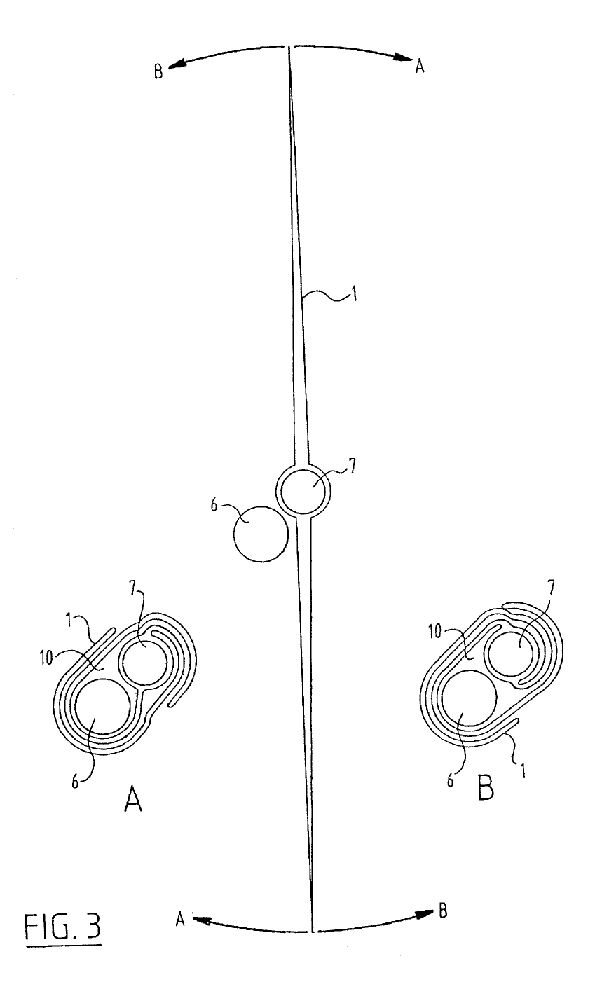

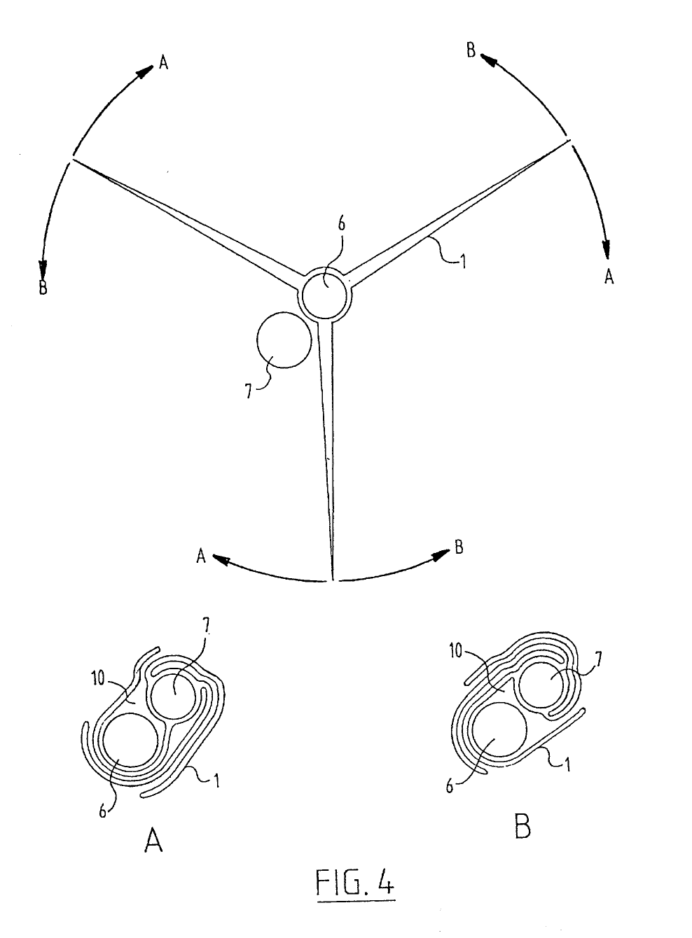

The folding method is illustrated in FIGS. 3 and 4. In FIGS. 3 and 4, the same reference numerals are used as in FIGS. 1 and 2. A balloon 1 with the stiffening wire 7 received therein is folded, in a manner which is known per se, to form a kind of propeller profile comprising two (FIG. 3), three (FIG. 4) or four (not shown) wings, depending on the diameter of the balloon. The guide wire 6 is arranged parallel to the stiffening wire 7. Finally, the wings are folded in anti-clockwise direction, indicated at B, or clockwise direction, indicated at A, to obtain the result indicated at A or the result indicated at B, respectively. The sectional views of FIGS. 3 and 4 clearly show at A and B that as a result of the special manner of folding the balloon 1, the guide wire 6 is received in a cavity 10 that has been obtained by folding the wings of the balloon 1 around the stiffening wire 7 and the guide wire 6. As a result, a very compact structure is obtained, which makes it possible to guide the balloon catheter through vessels and lumina.

It is noted that the invention is not limited to the illustrated preferred embodiment. Thus it is possible to have the guide wire 6 extend entirely through the second lumen 4 to the proximal end thereof.

What is claimed is:

1. A balloon catheter comprising a catheter tube having a first lumen and a second lumen, each lumen having a proximal end and a distal end, an inflatable balloon having a proximal end and a distal end, and a guide wire receivable in the second lumen, with the distal end of the first lumen opening into the balloon, characterized in that the distal end of the second lumen is open and positioned proximally of the proximal end of the balloon.

2. A balloon catheter according to claim 1, wherein the catheter includes a tip, and the distal end of the balloon is connected to the tip.

3. A balloon catheter according to claim 2, wherein the guide wire has a distal end, and the tip comprises a lumen for receiving the distal end of the guide wire.

4. A balloon catheter according to claim 1, wherein the catheter tube is provided with an opening which is in communication with the second lumen, which opening is positioned proximally of the distal end of the second lumen.

5. A balloon catheter according to claim 1, which further comprises a stiffening wire which is received in the first lumen.

6. A catheter having a proximal end and a distal end, comprising an inflatable balloon at the distal end of the catheter and a guide wire at least at the distal end of the catheter, characterized in that the guide wire is positioned free of and outside of the balloon at the distal end of the catheter in an inflated condition of the balloon.
